(19) United States
(12) Patent Application Publication
(10) Pub. No.: US 2009/0233301 A1
(43) Pub. Date: Sep. 17, 2009

(54) In Vitro System for Assessing Immune Activity Using Pig Immune Cells

(76) Inventor: Shing-Mou Lee, Danshuei Township (TW)

Correspondence Address: OCCHIUTI ROHLICEK & TSAO, LLP, 10 FAWCETT STREET, CAMBRIDGE, MA, 02138, US

(21) Appl. No.: 12/402,983

(22) Filed: Mar. 12, 2009

(30) Foreign Application Priority Data
Mar. 12, 2008 (TW) 097108773

(51) Int. Cl. C12Q 1/68; C12Q 1/02

(52) U.S. Cl. 435/6; 435/29

(57) ABSTRACT

An in vitro system in which immune cells of a non-immunized pig are used for assessing immunoactivity of a substance.

### CROSS-REFERENCE TO RELATED APPLICATION

This application claims the benefit of Taiwanese Patent Application No. 097108773, filed Mar. 12, 2008, the content of which is incorporated herein by reference in its entirety.

### BACKGROUND OF THE INVENTION

Many herb products and health foods help balance the immune system, thereby facilitating treatment of diseases associated with immune system deregulation. Therapeutic compounds, on the other hand, often elicit undesired immune responses, resulting in chronic immune disorders.

Human peripheral blood mononuclear cells (PBMCs) are routinely used for examining immune cell activities. They constitute a conventional in vitro system for identifying immune balancers or assessing the risk of a therapeutic compound for developing chronic immune disorders. This in vitro system has at least two disadvantages. First, its application is highly limited due to the scarcity of human PBMC resources. Second, given the diversity of human PBMC donors, results obtained from this system are almost invariably reproducible.

### SUMMARY OF THE INVENTION

The present invention features a method of assessing immunoactivity of a substance (e.g., a small molecule, a protein, an herbal product, or a lactobacillus lysate) with pig immune cells. This method includes: (i) providing immune cells of a non-immunized pig, (ii) contacting the immune cells with a substance of interest, (iii) examining an immune response induced by the substance, and (iv) assessing immunoactivity of the substance based on the immune response it elicits. The pig immune cells (e.g., PBMCs, CD4 T cells, CD8 T cells, natural killer T cells, B cells, natural killer cells, macrophages, or neutrophils) can be isolated from a Specific Pathogen Free pig or an immune deficiency pig. The immune responses to be examined can be cell proliferation, cell differentiation, and cell activity (e.g., cell activation, cytokine secretion, antibody secretion, phagocytosis, and cytotoxicity). The immune activity of the substance can be determined based on the immune response(s) it elicits. For example, a substance is determined to be an immune enhancer if it activates natural killer cells, increases phagocytosis of macrophages or neutropils, or improves cytotoxicity.

In one embodiment, the contacting step in the method described above is performed by contacting the pig immune cells with both a substance of interest and an immune stimulant (e.g., phytohemagglutinin, lipopolysaccharide, or an anti-CD3 antibody). If the level of an immune response elicited in the presence of both the substance and the stimulant is lower than that in the presence of the stimulant alone, it indicates that the substance is an immune suppressor.

The details of one or more embodiments of the invention are set forth in the description below. Other features or advantages of the present invention will be apparent from the following detailed description of several embodiments and also from the appended claims.

### DETAILED DESCRIPTION OF THE INVENTION

This invention relates to an in vitro system for assessing a substance's immune activity in immune cells isolated from a non-immunized pig. The term “non-immunized pig” used herein refers to a pig that has not been challenged by a man-made immunogenic composition that elicits an immune response against a target, such as a pathogen.

The immune cells used in this in vitro system can be PBMCs or a particular type of immune cells, e.g., T cells, B cells, natural killer cells, or macrophages. PBMCs can be purified from blood by a conventional method, e.g., Ficoll gradient centrifugation. Particular types of immune cells can be isolated from PBMCs or from thymus or bone marrow by conventional methods. See, e.g., Current Protocols in Immunology, John Wiley and Sons, Inc.

Any of the immune cells mentioned above are incubated in a suitable medium with a substance suspected of having a certain immune property. After a sufficient period of time, the cells and the culturing medium are collected and analyzed to determine immune responses elicited by the substance. The type of immune responses to be examined depends on the type of immune cells used or the particular immune property that the substance might have. Table 1 below lists exemplary immune cells and the immune responses that they involve.

TABLE 1
Type of Immune Cells and Associated Immune Responses

| Type of Immune Cells | Immune Response |
|---|---|
| PBMCs | Cell proliferation, Cell differentiation; |
| | Cell activation, Cytotoxicity, Cytokine |
| | secretion, Antibody secretion, |
| | Phagocytosis |
| CD4 T cells | Cell prolieration, Cell activation, |
| | Cytokine secretion |
| CD8 T cells | Cell prolieration, Cell activation, |
| | Cytokine secretion, Cytotoxicity |
| Natural Killer cells | Cytotoxicity, Cytokine secretion |
| B cells | Antibody secretion |
| Macrophages and Neutrophils | Phagocytosis, Cytokine secretion |

Various methods are available to examine immune responses, including those listed in Table 1 above. See, e.g., Current Protocols in Immunology, John Wiley and Sons, Inc. In one example, phenotyping is performed to examine a cell surface marker that is associated with a particular immune activity or a particular immune cell population. Immune cell surface markers (e.g., CD3, CD4, CD8, CD19, or CD56) and their association with particular types of immune activities or cell populations are well known in the art. See, e.g., Abbas et al., Cellular and Molecular Immunology, 4^{th} Ed., W.B. Saunders, Appendix II at pages 500-514. Presence of a cell surface marker and the level of the marker are indicative of exhibition/level of the associated immune activity or existence/percentage of the particular cell population. In another example, quantitative polymerase-chain reaction is performed to determine the mRNA level of a cytokine. Cytokines are well known to be associated with immune cell activation and immune responses. For example, IL-2 is an indicator of T cell proliferation. As another example, certain cytokines (e.g., TNF-alpha) play important roles in inflammation and others (e.g., IL-12) suppress inflammation. Thus, a substance's ability to increase the level of IL-2 indicates that it is a T cell activator and its ability to increase the level of a pro-inflammatory cytokine or an anti-inflammatory cytokine indicates that it may induce or suppress inflammation.

The in vitro system disclosed herein can be applied to identify a substance that has a particular immune property, such as enhancing, balancing, or suppressing immunity. To identify an immune enhancer, a candidate substance is examined for its ability to elicit immune responses indicative of immunity reinforcement, e.g., increasing phagocytosis of macrophages or neutrophils, and improving cytotoxicity of CD8 T cells, natural killer cells, or natural killer T cells. A substance capable of eliciting one or more of such immune responses is determined to be an immune enhancer. To identify an immune suppressor, a candidate substance is examined for its ability to decrease an immune response induced by an immune stimulant. A substance exhibiting this activity is determined to be an immune suppressor.

This in vitro system, using pig immune cells, has at least two advantages. When pigs having the same genetic background are used as immune cell donors, results obtained from this system are highly reproducible. In addition, this system is particularly useful for large scale screening for substances that have desired immune activities, given the abundance of pig immune cells.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### EXAMPLE 1

### Natural Killer Cell Activity in Pig Peripheral Blood Mononuclear Cells

Blood was drawn from a SPF pig and the peripheral blood mononuclear cells (PBMCs) were purified by Ficoll density centrifugation. The PBMCs were cultured in a RPMI 1640 medium supplemented with 10% fetal bovine serum at 37° C. in the absence or presence of IL-2 (12.5, 25, 50, or 100 ng/ml).

K-562 cells, sensitive to natural killer (NK) cells, were cultured in an Iscoves Modified Dulbeccos Medium supplemented with 10% FBS and 4 mM L-glutamine (the complete medium) at 37° C. for 24 hours. These cells were stained with 5 μm carboxyfluorescein succinimidyl ester (CFSE), incubated at 37° C. for 15 minutes, and then washed with the complete medium.

The PBMCs (5×10⁶/ml) and the CFSE-stained K-562 cells (1×10⁵/ml) were mixed at various ratios (12.5:1, 25:1, and 50:1 by cell number) and cultured at 37° C. for 4 hours. The cells were collected, washed with 1×HBSS, treated with 20 μg/ml propidium iodide (PI) for 10 minutes and then examined with a flow cytometer (Beckman Coulter) to determine the numbers of live and dead cells. The percentage of dead cells is designated PI %. K-562 cells alone were used as a blankcontrol and K-562 cells treated with Nonident P-40 were used as a positive control. The rate of natural killer cell cytotoxicity was calculated by the following formula:

Rate of NK cell cytotoxicity=(PI1% of cells treated by IL-2−PI % of blank control)/(PI % of positive control−PI % of blank control)×100%

As shown in Tables 2 and 3, the pig PBMCs exhibited NK cell cytotoxicity when interacted with K-562 cells. Increasing the ratio between the PBMCs and K-562 cells resulted in greater NK cell cytotoxicity.

TABLE 2
Rate of Cell Death

| | | | PBMCs:K-562 | | |
|---|---|---|---|---|---|
| PI % | | PBMCs | 12.5:1 | 25:1 | 50:1 |
| IL-2 | 0 | 7.59 | 6.01 | 10.21 | 14.7 |
| (ng/ml) | 12.5 | 4.09 | 4.42 | 7.59 | 11.09 |
| | 25 | 4.66 | 4.97 | 10.02 | 13.17 |
| | 50 | 5.41 | 5.49 | 7.08 | 10.08 |
| | 100 | 5.62 | 5.97 | 7.59 | 9.58 |

TABLE 3
Rate of NK Cell Cytotoxicity

| Rate | | | | | |
|---|---|---|---|---|---|
| of NK Cell | | | PBMCs:K-562 | | |
| Cytotoxicity % | | PBMCs | 12.5:1 | 25:1 | 50:1 |
| IL-2 | 0 | — | −1.70 | 2.83 | 7.69 |
| (ng/ml) | 12.5 | — | 0.34 | 3.65 | 7.30 |
| | 25 | — | 0.30 | 5.62 | 8.92 |
| | 50 | — | 0.08 | 1.76 | 4.94 |
| | 100 | — | 0.37 | 2.08 | 4.48 |

### EXAMPLE 2

### Immunoactivity Examination Using both Human and Pig PBMCs

### (i) Cell Proliferation

PBMCs were isolated from both human and SPF pig blood by Ficoll density centrifugation. 50 μl of the human or pig PBMCs (4×10⁶/ml) were placed in a 96-well plate, mixed with 50 μl Phytohaemagglutinin (PHA; 2.5 μg/ml), and incubated at 37° C. for 69 hours.

The cells were then mixed with 10 μl alamarBlue for three hours. Their optical densities were examined afterwards. As shown in Table 4 below, both pig and human PBMCs proliferated when stimulated by PHA. The pig PBMCs required a lower PHA concentration for cell proliferation then the human PBMCs.

TABLE 4
Proliferation of Pig and Human PBMCs Treated with PHA

Reduced alamarBlue %

| PHA Conc. (ug/ml) | Pig PBMCs | Human PBMCs |
|---|---|---|
| 0 | 25.6 | 41.1 |
| 0.16 | 26.9 | 40.9 |
| 0.31 | 30 | 45 |
| 0.63 | 51.9 | 55.1 |
| 1.25 | 52.5 | 62.2 |
| 2.5 | 54.7 | 61 |
| 5 | 57.7 | 63.5 |

### (ii) Cytokine Secretion

The pig and human PBMCs mentioned above were placed in a six-well plate (5×10⁶ per well) and incubated with PHA at various concentrations at 37° C. for 7 hours (human PBMCs) or 16 hours (pig PBMCs). RNAs were then extracted from these cells using the RNeasy® Mini kit (Qiugen) and subjected to reverse transcription and then multiplex polymerase chain reaction to examine the expression levels of cytokines IL-2, IFN-γ, IL-4, and IL-10.

The results indicate that PHA stimulated secretion of the above listed cytokines in both pig and human PBMCs. The concentrations of PHA did not significantly affect the cytokine levels.

(iii) NK Cell Cytotoxicity

The pig and human PBMCs mentioned above were incubated with or without IL-2 (100 ng/ml) at 37° C. for 16 hours and then mixed with K-562 cells at various ratios. The mixed cells were incubated at 37° C. for 4 hours, stained with propidium iodide for 10 minutes, and analyzed with a flow cytometer.

TABLE 5
Rates of NK Cell Cytotoxicity in Human PBMCs

| | PBMC:K-562 | Rate of NK Cell Cytotoxicity % |
|---|---|---|
| IL-2 Untreated | 12.5:1 | 8.8 |
| | 25:1 | 19.7 |
| | 50:1 | 52.6 |
| IL-2 Treated | 12.5:1 | 60.6 |
| | 25:1 | 86.4 |
| | 50:1 | 89.3 |

TABLE 6
Rates of NK Cell Cytotoxicity in Pig PBMCs

| | PBMC:K-562 | Rate of NK Cell Cytotoxicity % |
|---|---|---|
| IL-2 Untreated | 12.5:1 | 1.1 |
| | 25:1 | 2.6 |
| | 50:1 | 4.2 |
| | 200:1 | 9.7 |
| IL-2 Treated | 12.5:1 | 4.7 |
| | 25:1 | 11.5 |
| | 50:1 | 22.5 |
| | 200:1 | 36.5 |

Like the human PBMCs, the pig PBMCs also exhibited NK cell cytotoxicity when stimulated by IL-2. See Tables 5 and 6 above.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of assessing immunoactivity of a substance, comprising
providing immune cells of a non-immunized pig,
contacting the immune cells with a substance,
examining an immune response of the immune cells induced by the substance, the immune response being cell proliferation, cell differentiation, or cell activity, and
assessing immunoactivity of the substance based on the immune response induced thereby.

2. The method of claim 1, wherein the immune response being examined is cell activity selected from the group consisting of cell activation, cytokine secretion, antibody secretion, phagocytosis, and cytotoxicity.

3. The method of claim 1, wherein the immune cells are peripheral blood mononuclear cells (PBMCs).

4. The method of claim 1, wherein the immune cells are T cells, B cells, macrophages, dendritic cells, neutrophils, or natural killer cell.

5. The method of claim 2, wherein the immune cells are macrophages or neutrophils and the cell activity being examined is phagocytosis of the macrophages or neutrophils.

6. The method of claim 2, wherein the immune cells are CD8⁺ T cells, natural killer (NK) cells, or NK T cells and the cell activity being examined is cytotoxicity of the CD8⁺ T cells, NK cells, or NK T cells.

7. The method of claim 2, wherein the immune cells are B cells and the cell activity being examined is antibody secretion.

8. The method of claim 1, wherein the pig is a Specific Pathogen Free pig.

9. The method of claim 1, wherein the immune response is examined by phenotyping.

10. The method of claim 2, wherein the cell activity being examined is cytokine secretion, which is determined by a protein or mRNA level of a cytokine.

11. The method of claim 1, wherein the substance is a small molecular, a protein, an herbal extract, or a lactobacillus lysate.

12. The method of claim 3, wherein the PBMCs are isolated from a Specific Pathogen Free pig.

13. The method of claim 3, wherein the immune response is examined by phenotyping.

14. The method of claim 3, wherein the immune response being examined is cell activity selected from the group consisting of cell activation, cytokine secretion, antibody secretion, phagocytosis, and cytotoxicity.

15. The method of claim 14, wherein the cell activity being examined is phagocytosis of macrophages or neutrophils.

16. The method of claim 14, wherein the cell activity being examined is cytotoxicity of CD8⁺ T cells, NK T cells, or NK cells.

17. The method of claim 14, wherein the cell activity being examined is cytokine secretion, which is determined by a protein or mRNA level of a cytokine.

18. The method of claim 3, wherein the substance is a small molecular, a protein, an herbal extract, or a lactobacillus lysate.

19. The method of claim 1, wherein the substance is determined to be an immune enhancer if it activates natural killer cells, increases phagocytosis of macrophages or neutrophils, or enhances cytotoxicity.

20. The method of claim 1, wherein the method is carried out in high throughput format.

21. A method of identifying an immune suppressor, comprising
providing immune cells of a non-immunized pig,
contacting the immune cells with an immune stimulant and a substance,
examining an immune response of the immune cells induced by both the stimulant and the substance, the immune response being cell proliferation, cell differentiation, or cell activity, and
assessing whether the substance is an immune suppressor, wherein a lower level of the immune response relative to that induced by the stimulant alone indicates that the substance is an immune suppressor.

22. The method of claim 21, wherein the immune cells are peripheral blood mononuclear cells (PBMCs).

23. The method of claim 21, wherein the immune cells are T cells, B cells, macrophages, dendritic cells, neutrophils, or natural killer cells.

24. The method of claim 21, wherein the substance is a small molecular, a protein, an herbal extract, or a lactobacillus lysate.

25. The method of claim 21, wherein the immune response being examined is cell activity selected from the group consisting of cell activation, cytokine secretion, antibody secretion, phagocytosis, and cytotoxicity.
